(19) United States
(12) Patent Application Publication
(10) Pub. No.: US 2025/0100115 A1
(43) Pub. Date: Mar. 27, 2025

(54) Spring Clutch For Power Torque Wrench

(71) Applicant: K2M, Inc., Leesburg, VA (US)

(72) Inventors: Amir Ali Sharifi-Mehr, Bloomingdale, NJ (US); Brandon Moore, Leesburg, VA (US); Mary Patricia Hamburger, Stone Ridge, VA (US); Jason Noel Gantick, Purcellville, VA (US)

(21) Appl. No.: 18/888,336

(22) Filed: Sep. 18, 2024

Related U.S. Application Data
(60) Provisional application No. 63/539,848, filed on Sep. 22, 2023.

(51) Int. Cl. B25B 23/147; A61B 17/16; B25B 23/14; B25F 5/00

(52) U.S. Cl. CPC B25B 23/147; A61B 17/1613; B25B 23/141; B25F 5/001

(57) ABSTRACT

A torque limiter having a clutch assembly including a quick connect drive, a one-piece spring clutch, and a torque shaft. The quick connect drive includes at least a first set of gear teeth. The one-piece clutch includes a top surface defining a second set of gear teeth and a bottom surface defining a third set of gear teeth, and the torque shaft includes a fourth set of gear teeth. The clutch assembly is assembled such that the first set of gear teeth engage the second set of gear teeth and the third set of gear teeth engage the fourth set of gear teeth.

### CROSS-REFERENCE TO RELATED APPLICATIONS

This application claims the benefit of the filing date of U.S. Provisional Patent Application No. 63/539,848 filed Sep. 22, 2023, the disclosure of which is hereby incorporated herein by reference.

### BACKGROUND

During a spinal fusion surgery involving pedicle screws, a spinal surgeon could be required to tighten anywhere from 6 to more than 30 locking caps/set screws. As a result, spinal surgeons often suffer from repeated action injuries. To prevent this, as well as to improve surgical efficiency, power tools have become more desirable than manual tools. To improve the safety and efficacy of power tools used during surgical operations, a torque limiting instrument such as a power torque wrench is often used to prevent transferring too much applied torque to the patient's bone structure (e.g., the spine). These power torque wrench solutions are often low cost, reliable, and are compatible with power tools already available in the operating room.

Many current torque limiting instruments contain coil springs or wave springs to prevent providing too much axial force and/or involve complex internal structures with multitudes of components that lend themselves to mechanical failures. Often, the complexity of the internal structures makes repair or replacement of the internal components difficult. Moreover, current manual torque wrenches are often used with a separate anti-torque keys to prevent transfer of torque to the patient's spine. This creates the need for additional instruments during surgery. Additionally, existing inventions often include exposed clutch components and gears that rotate with the shaft, increasing the likelihood of complications during the surgery as the users or the users' clothing come into contact with the rotating units during operation.

Further improvements are desired in the field of surgical tools for torque limiting instruments that provide increased safety and durability.

### BRIEF SUMMARY

In one aspect, the present disclosure is directed to a torque limiting mechanism including a quick connect drive or drive shaft, a torque shaft, a single-member spring clutch, and a housing. Often coupled to a power hand drill, the torque limiting mechanism can prevent the torque applied to instruments, such as spinal pedicle screws with locking caps, from transferring to the patient's spine. The quick connect drive and torque shaft have gear teeth that engage with the single-member spring clutch to transfer torque to a bone screw. The single-member spring clutch defines a single unified structure that has two clutch faces with gear teeth that are arranged for ratcheting movement between the torque shaft and quick connect drive when a maximum torque limit is exceeded. Characteristics of the single-member spring clutch determine the maximum torque of the torque limiting mechanism as a whole. The maximum torque of the mechanism can be modified by altering the material properties (e.g., yield strength, tensile strength, modulus of elasticity, etc.) and/or the geometrical dimensions of the single-member clutch. The single-member spring clutch transfers torque from the quick connect drive to the torque shaft up to a set maximum torque. When the maximum torque is achieved, the single-member spring clutch will actuate (i.e., compress, flex or deform in some manner) and stops transferring torque to the torque shaft as the single-member ratchets (i.e., slips) between the quick connect drive and torque shaft. In this manner, the single-member spring clutch prevents overtightening of screws being tightened with a power drill.

The housing covers and holds together the single-member spring clutch, the quick connect drive, and the torque shaft. The quick connect drive rotatably connects with the housing via pin inserted through the housing and thereby holds the single-member spring clutch and a proximal end portion of the torque shaft within the housing. In other words, the positions of the torque shaft, the single-member spring clutch, and the quick connect drive are fixed with respect to movement along the longitudinal axis of the torque limiter but can freely rotate about that axis due to the rotatable connection between the quick connector drive and the housing. This allows the housing to remain stationary while the single-member spring clutch, quick connect drive, and torque shaft rotate therein. Because the housing is stationary, it may be coupled to a non-rotating portion of the power drill and protect users from encountering the single-member spring clutch during operation. Additionally, the housing may include a component by having an anti-torque key extending distally toward the tip of the torque shaft.

In another aspect, a torque limiter may include a quick connect, a one-piece clutch, and a torque shaft. The quick connect drive may have first gear teeth, the one-piece clutch may have a top surface defining second gear teeth and a bottom surface may define third gear teeth, and the torque shaft may have fourth gear teeth such that the first gear teeth engage the second gear teeth and the third gear teeth engage the fourth gear teeth. The one-piece clutch along with the second gear teeth and the third gear teeth may define one monolithic body. In such embodiments, the torque limiter may further include a housing disposed around portions of the quick connect drive, the torque shaft, and the one-piece clutch. In such embodiments, the housing may encase the first, second, third, and fourth gear teeth. In such embodiments, the housing may further include an anti-torque key. In such embodiments, the housing may include slots disposed radially on a proximal edge of the housing. In such embodiments, housing may include pin holes that are disposed around the housing and configured to align with a channel in the quick connect drive, the channel being adjacent to the first gear teeth. In such embodiments, the torque limiter may further include pins that are disposed partially within the channel of the quick connect and partially within the pin holes of the housing. In other embodiments, each of the pin holes aligns with one of the slots.

In other embodiments, each of the pins sit below an outer surface of the housing that surrounds each of the pin holes. In other embodiments, the pins may include a head portion that sits above the outer surface of the housing, the head portion configured to be manually rotated such that the pin can be threaded into and out of the pin holes. The slots may be configured to engage with segments of a power drill. The one-piece clutch is configured to transfer an applied torque from the quick connect drive to the torque shaft up to a maximum torque. In such embodiments, the one-piece clutch may be configured to compress under an amount of torque that exceeds the maximum torque. The one-piece clutch may be configured to compress and expand like a compression spring. The compression of the one-piece clutch may be configured to allow the second gear teeth or the third gear teeth to slip with respect to the first or fourth gear teeth, respectively. The one-piece clutch may be additively manufactured.

In other embodiments, the one-piece clutch may further include a plurality of pillars integrally formed between a plurality of layers that extend perpendicular to a longitudinal axis of the torque limiter. The plurality of pillars may extend parallel to the longitudinal axis of the torque limiter. Two of the plurality of pillars may be located between each of the plurality of layers. In such embodiments, the two of the plurality of pillars are aligned with each other in a direction perpendicular to the longitudinal axis of the torque limiter. A position of the two of the plurality of pillars may alternate along a circumference of the one-piece clutch between each of the layers. In some embodiments, the two of the plurality of pillars located between a first layer, a second layer, a third layer, a fourth layer, a fifth layer, and a sixth layer may be positioned such that the pillars between the first and second layers are separately aligned along the circumference of the one-piece clutch with the pillars between the third and fourth layers and the fifth and six layers, respectively.

In other embodiments, the position of the two of the plurality of pillars along the circumference of the one-piece clutch alternates approximately 90 degrees around the circumference of the one-piece clutch between each of the plurality of layers. Some of the plurality of pillars may connect to the plurality of layers to the second gear teeth and the third gear teeth. The one-piece clutch may be a cylinder and may further include a central aperture extending through both the second and third teeth. A portion of the quick connect drive may be disposed within the central aperture. The first and second gear teeth may each have a plurality of contact surfaces that conform to each other, and the third and fourth gear teeth may each have a plurality of surfaces that conform to each other. The plurality of contact surfaces and the plurality of surfaces are oriented diagonal to the longitudinal axis of the torque limiter. The quick connect drive includes a V-shaped groove extending around the quick connect drive. The quick connect drive may include an alignment shaft extending from the first gear teeth along the longitudinal axis of the torque limiter, the alignment shaft may be configured to extend through the central aperture of the one-piece clutch and into a central hole in the torque shaft.

In other embodiments, a surgical instrument may include the torque limiter and a power handpiece (e.g., a hand-held power drill). In such embodiments, the quick connect drive may engage the powered handpiece, and the housing may engage the powered handpiece.

In accordance with another aspect, a kit may include a quick connect, a torque shaft, a plurality of one-piece clutches. The quick connect may have first teeth, the torque shaft may have second teeth, and each of the plurality of one-piece clutches may have a top clutch face defining third teeth and a bottom clutch face defining fourth teeth such that the first teeth engage the third teeth and the second teeth engage the fourth teeth, each one-piece clutch may permit a different maximum torque. The kit may further include a powered handpiece. The kit may further include a housing configured to enclose a portion of the quick connect drive, the torque shaft, and one of the plurality of one-piece clutches in an assembled arrangement. In other embodiments, the kit may further include pins that engage the housing and the quick connect drive such that the quick connect drive, the torque shaft, and one of the plurality of one-piece clutches remain in contact with each other.

In accordance with another aspect, a screw or a locking cap may be tightened by a process. In the process, a tip of a torque limiter may be inserted into a head of the screw or locking cap, and the torque limiter may include at least a torque shaft, a one-piece clutch, and a quick connect drive at least partially disposed within a non-rotating housing. Next, torque may be transferred from a powered handpiece to the screw or locking cap via the torque limiter, the powered handpiece and the torque limiter are connected via the quick connect drive. Subsequently, the non-rotating housing may be grasped while actuating the powered handpiece and torque limiter.

The process may further include the one-piece clutch being compressed by applying an amount of torque that exceeds a maximum torque threshold. The process may additionally include the quick connect drive being inserted into the powered handpiece and the non-rotating housing being interlocked with the powered handpiece.

In accordance with another aspect, a surgical instrument for tightening surgical hardware may include a handle, a torque shaft having a cylindrical structure, a quick connect drive, and a break-away support. The quick connect drive may be disposed between the handle and the torque shaft. The break-away support may be disposed around the quick connect drive and fixed to the handle and the torque shaft. In such embodiments, the quick connect drive may extend from the torque shaft and may be configured to engage the handle. The handle may define a slot configured to receive a portion of the quick connect drive. In such embodiments, the quick connect drive may include a tapered end that is disposed within the slot of the handle. In other embodiments, the quick connect drive may extend from the handle and may be configured to engage with the torque shaft. The torque shaft may define a slot configured to receive a portion of the quick connect drive. In such embodiments, the quick connect drive may include a plurality of spring-loaded rollers configured to resist rotation between the torque shaft and the quick connect drive.

In accordance with yet another aspect, a surgical instrument for tightening surgical hardware may include a handle, a sleeve connected to and extending form the handle, and a break-away torque shaft. The break-away torque shaft may have a plurality of engagement members disposed within the sleeve, and each of the engagement members may be configured to be inserted into a head of a screw. In such embodiments, the plurality of engagement members may be connected in series by a narrow segment between each engagement member.

### BRIEF DESCRIPTION OF THE DRAWINGS

A more complete appreciation of the subject matter of the present disclosure and the various advantages thereof may be realized by reference to the following detailed description which refers to the accompanying drawings, in which:

FIG. 1 is a perspective view of a torque limiter coupled to a power drill in accordance with an embodiment of the present disclosure;

FIG. 2 is a perspective view of the torque limiter and the power drill of FIG. 1, the torque limiter decoupled from the power drill;

FIG. 3 is an exploded view of the torque limiter of FIG. 1 without a housing component;

FIG. 4 is an enlarged view of the torque limiter of FIG. 1 with the housing component shown in a transparent fashion;

FIG. 5 is an exploded view of the torque limiter of FIG. 1 with the housing component;

FIG. 6 is a perspective view of a quick connect drive of the torque limiter of FIG. 3;

FIG. 7 is a perspective view of a torque shaft of the torque limiter of FIG. 3;

FIG. 8 is a perspective view of a one-piece clutch of the torque limiter of FIG. 3;

FIG. 9 is a cross sectional view of the one-piece clutch of the torque limiter of FIG. 1 taken along line A-A;

FIG. 10 is a cross sectional view the torque limiter and power drill of FIG. 1 taken along line A-A;

FIG. 11 is a cross sectional view of the torque limiter of FIG. 5;

FIG. 12 is a perspective view of a torque limiter decoupled from a power drill in accordance with another embodiment of the present disclosure;

FIG. 13 is an exploded view of the torque limiter of FIG. 12;

FIG. 14 is a perspective view of the torque limiter of FIG. 12;

FIG. 15 is a cross sectional view of the torque limiter of FIG. 12 taken along line B-B;

FIG. 16 is a perspective view of the torque limiter of FIG. 12;

FIG. 17 is a perspective view of the torque limiter of FIG. 12 engaging a locking cap;

FIG. 18 is a side view of a handle and a torque limiter in accordance with another embodiment of the present disclosure;

FIG. 19 is an exploded view of the torque limiter of FIG. 18;

FIG. 20 is a cross sectional view of the torque limiter of FIG. 18 along line C-C;

FIG. 21 is a cross sectional view of torque limiter of FIG. 20 along line D-D; and

FIG. 22 is a cross sectional view of a torque limiter in accordance with another embodiment of the present disclosure.

### DETAILED DESCRIPTION

As used herein unless stated otherwise, the term “anterior” means toward the front part of the body, and the term “posterior” means toward the back part of the body. When referring to specific directions in the following discussion of a certain device, the terms “proximal” and “distal” are to be understood in regard to the device's orientation and position during exemplary application to human body. Thus, the term “proximal” means closer to the operator or in a direction toward the operator, and the term “distal” means more distant from the operator or in a direction away from the operator. In addition, the terms “about,” “generally,” and “substantially” are intended to mean that deviations from absolute are included within the scope of the term so modified.

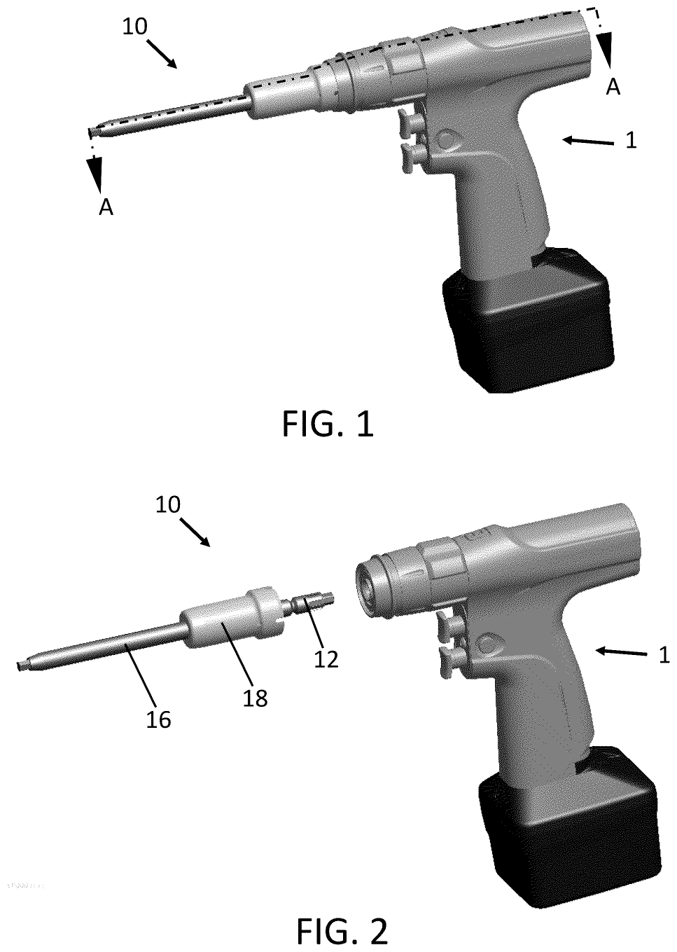

In a first aspect, the present disclosure relates to a torque limiter 10 designed to prevent over-torquing (i.e., over-tightening) locking caps and/or screws and configured to attach to a powered handpiece such as a handheld power drill 1 suited for surgical operations, as shown in FIGS. 1-2. Torque limiter 10 includes a clutch assembly and a housing 18 that covers at least a portion of the clutch assembly and is configured to couple with power drill 1 (discussed in more detail below).

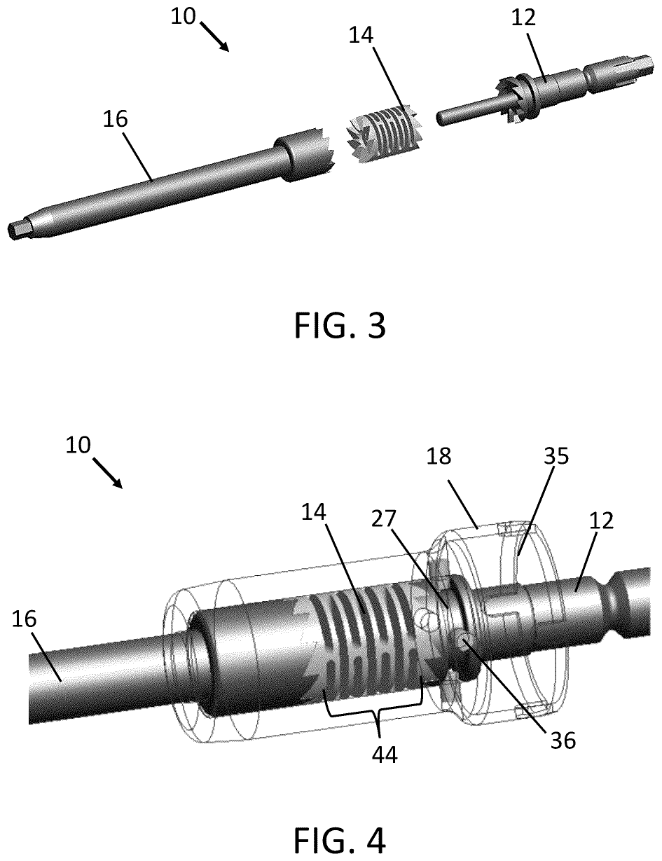

In one embodiment, as shown in FIGS. 3-11, the clutch assembly of torque limiter 10 includes a quick connect 12, a one-piece clutch 14, and a torque shaft 16. Quick connect 12 is a drive shaft that includes a proximal portion, an intermediate portion, and a distal portion. The proximal portion has a first end 21 defining hexagonal shape configured to be inserted into a power drill and a groove 23 that is adjacent to first end 21. Groove 23 has a V-shaped profile that extends radially around quick connect 12. The distal portion includes an alignment shaft 29 that extends from the intermediate portion. Alignment shaft 29 is configured to extend through one-piece clutch 14 and into torque shaft 16.

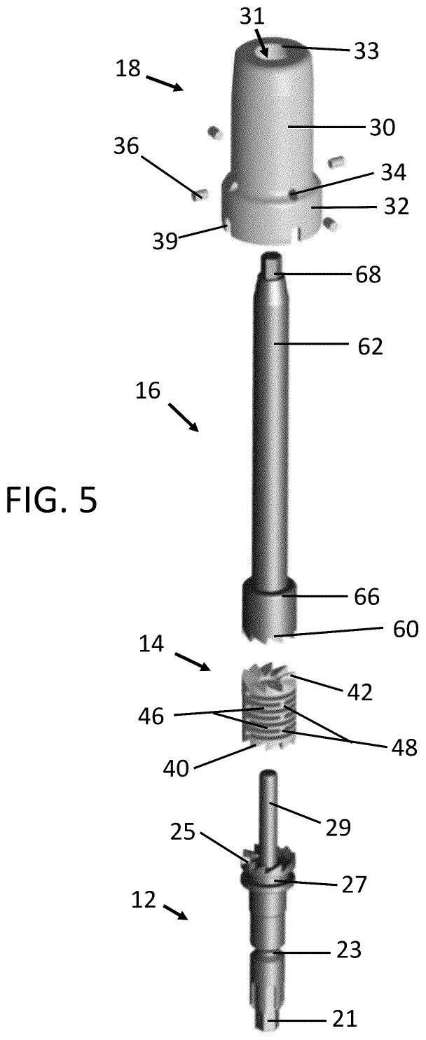

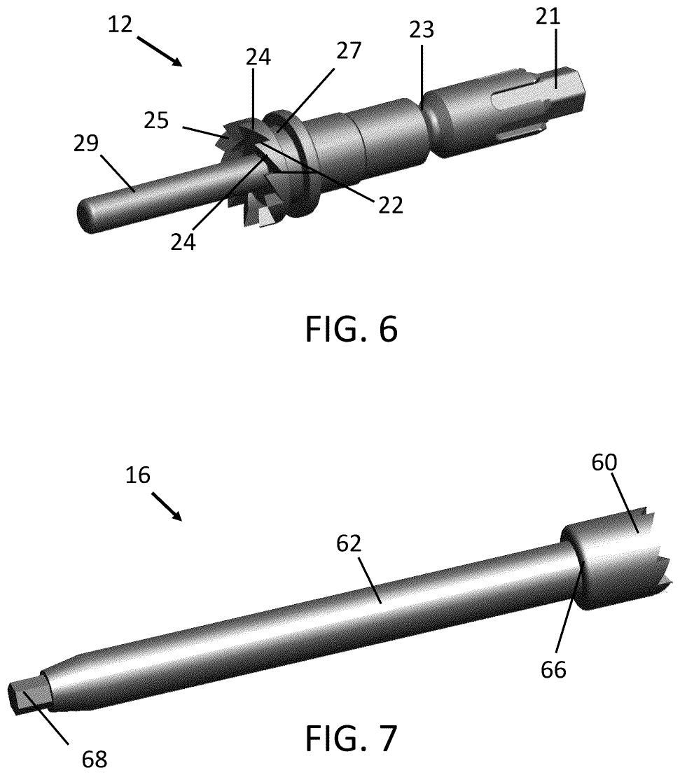

The intermediate portion of quick connect 12 includes a first gear 25 and a channel 27 having a U-shaped profile, as shown in FIGS. 5-6. Channel 27 is immediately adjacent to first gear 25 on the proximal side and extends radially around quick connect 12. Channel 27 is configured to receive pins 36 such that the pins can fit into and movement circumferentially around the channel (discussed in more detail below). First gear 25 has a plurality of planar surfaces 24 that face in a distal direction and are oriented diagonal to the longitudinal axis of quick connect 12. Additionally, a first gear 25 has a plurality of stop surfaces 22 that are parallel to the longitudinal axis of quick connect 12. The stop surfaces face in a counterclockwise direction when viewed from the perspective of the alignment shaft 29. Gear teeth of first gear 25 are planar surfaces 24 and stop surfaces 22.

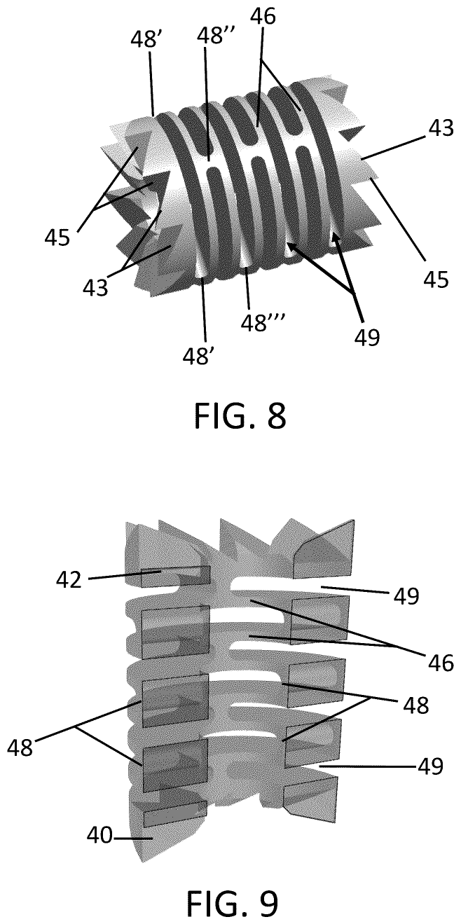

One-piece clutch 14 is a single integrated structure that includes a second gear 40, a third gear 42, a flexible region 44 that is disposed between the first and second gear, and a hollow annulus 41 that extends therethrough along the longitudinal axis of the one-piece clutch, as best shown in FIGS. 8-9. Second gear 40 and third gear 42 include a plurality of angled surfaces 43 that are oriented diagonal to the longitudinal axis of one-piece clutch 14. Each angled surface 43 is located between two non-angled surfaces 45 that are oriented parallel to the longitudinal axis of one-piece clutch 14, the angled and non-angled surfaces are the gear teeth of second gear 40 and third gear 42. When torque limiter 10 is assembled, second gear 40 faces in a proximal direction toward quick connect 12 and third gear 42 faces in a distally direction toward torque shaft 16. The gear teeth of second gear 40 are the inverse structure of the gear teeth of first gear 25 such that the first and second gear fit tightly together, as shown in FIG. 4.

Flexible region 44 of one-piece clutch 14 comprises layers 46 that are connected together by a plurality of pillars 48. Layers 46 are spaced apart from each other along the longitudinal axis of one-piece clutch 14 and thereby create several gaps 49. In each gap 49, a pair of pillars 48 are aligned and positioned directly opposite to each other along a direction perpendicular to the longitudinal axis of one-piece clutch 14. The position of each pair of pillars 48 alternates at every stage within layers 46 such that no two consecutive pairs of the pillars are aligned with respect to their position along the circumference of one-piece clutch 14. In other words, a first, third, fifth, and seventh pair of pillars 48 (i.e., odd pillar set) are all aligned along the circumference of the one-piece clutch 14, and a second, fourth, and sixth pair of pillars 48 are all aligned with each other in a similar manner at a position 90 degrees away from the odd pillar set along the circumference of one-piece clutch 14. For example, as shown in FIGS. 8, a first pair of pillars 48′ will positioned 90 degrees away from a second pair of pillars 48″ and aligned with a third pair of pillars 48′″, the second pair of pillars being located between the first and third pairs of pillars. This alternating placement of pillars 48 continues for the remainder of flexible region 44 and subdivides gaps 49 between layers 46. One-piece clutch 14 may include any number of pillars 48, gaps 49, and/or layers 46 of various dimensions and materials depending on the desired parameters of the one-piece clutch (e.g., maximum torque threshold, dimensions, etc.). One-piece clutch 14 is fabricated as a single part (i.e., a single unit defining one unified structure). Additionally, one-piece clutch 14 may be fabricated by additive manufacturing (i.e., 3D printing), e.g., but not limited to, binder jetting, selective laser melting (SLM), selective laser sinter (SLS), electron beam melting (EBM), direct metal laser sintering (DMLS), etc., and by other fabrication methods that utilize computer aided manufacturing (CAM) techniques. Furthermore, one-piece clutch 14 may be formed by fabrication techniques described in U.S. Pat. Nos. 4,863,538, 5,017,753, 5,076,869, 4,944,817, 10,614,176, 11,534,307, 11,737,880, and 7,537,664, the entire disclosures of which are incorporated by reference.

Referring to FIGS. 8-9, pillars 48 of one-piece clutch 14 act as support structures that resist deformation, particularly deformation caused by axial compression and/or applied torque. However, gaps 49 between the pairs of pillars 48 allow one-piece clutch 14 to temporarily compress when external forces, e.g., torsional and/or axial forces, are applied to the one-piece clutch. The material characteristics and the geometry of the structure of flexible region 44 also gives one-piece clutch 14 spring-like characteristics such that the flexible region can spring back its original form after experiencing brief deformation (i.e., elastic deformation). One-piece clutch 14 is made from material(s) with such characteristics such as, but not limited to, stainless steel, other alloys of steel, or plastics including acetal copolymer or acetal homopolymer, that will facilitate this spring-like response when external forces are applied. When higher maximum torque limits are required, one-piece clutch 14 may be fabricated from stiffer materials such as stainless steel, while plastics may be used for fabrication of the one-piece clutch when lower maximum torque limits are needed. The stiffness of the one-piece clutch 14 may be controlled and adjusted by material selection and adjusting structural dimensions (e.g., thicker or thinner pillars 48). One-piece clutch 14 is designed to maintain a general form while torsional forces are applied without deforming until a maximum torque is reached. Once the maximum torque is exceeded, flexible region 44 will momentarily deform or flex and cause the gear teeth of second gear 40 (or third gear 42 depending on the direction of rotation) to slip along the gear teeth of first gear 25 of quick connect 12 (or fourth gear 60 of torque shaft 16, discussed below), which interrupts torque being transferred from torque limiter 10 to a bone screw. In this manner, one-piece clutch 14 can prevent overtightening by limiting the amount of torque applied to a bone screw or locking cap when inserted or tightened by a power drill coupled to torque limiter 10, particularly when being final tightened. The slippage facilitated by clutch 14 can also act to provide an indication to the user that a maximum torque has been applied. This can be an audible sound created by the slippage, a feeling of lack of force in the powered handpiece, or the like.

Torque shaft 16 includes a fourth gear 60, a rod 62, and an alignment hole 64 that extends down the center of the torque shaft from a proximal end and through the center of the fourth gear. Fourth gear 60, like the other gears, includes a plurality of angled surfaces that are diagonal to the longitudinal axis of torque shaft 16 that make up the gear teeth of the fourth gear. Similar to the corresponding structure of the gear teeth of the first gear 25 and the gear teeth of the second gear 40, the gear teeth of fourth gear 60 are the inverse structure of the gear teeth of third gear 42 such the third and fourth gear fit closely together, as shown in FIG. 4. Rod 62 extends from fourth gear 60 to a tip 68, the rod having a smaller diameter than the outer diameter of fourth gear such that a shoulder 66 is formed on the distal side of the fourth gear. Tip 68 has a hexagon shape to be inserted and engaged with a hexalobular screw head. In other embodiments, tip 68 may be of any variety of shapes that conform to a screw head such as flathead, Philips, Frearson, etc. Additionally, tip 68 may be modular and removable from torque shaft 16 to allow multiple tool tips to be utilized.

In some embodiments, torque limiter 10 includes housing 18 that is designed to enclose a portion of clutch assembly, particularly one-piece clutch 14, and is configured to engage with various power drills, such as power drill 1. Housing 18 includes a cover portion 30, a cap portion 32 having slots 39 disposed radially around a proximal edge, and pin holes 34 that are evenly spaced around the circumference of the cover portion and immediately adjacent to the cap portion. Slots 39 are configured to engage with nubs on a power drill such that the power drill prevents the rotation of housing 18 while the internal components of torque limiter 10 are rotating. Cover portion 30 and cap portion 32 define a cavity 31 having a first opening 33 and second opening 35 located at opposite ends of the cavity, the first opening configured to receive rod 62 of torque shaft 16 and the second opening configured to receive other portions of torque limiter 10, including clutch 14. First opening 33 has a smaller diameter than the inner diameter of cavity 31 such that an inner shoulder 37 is formed around the first hole.

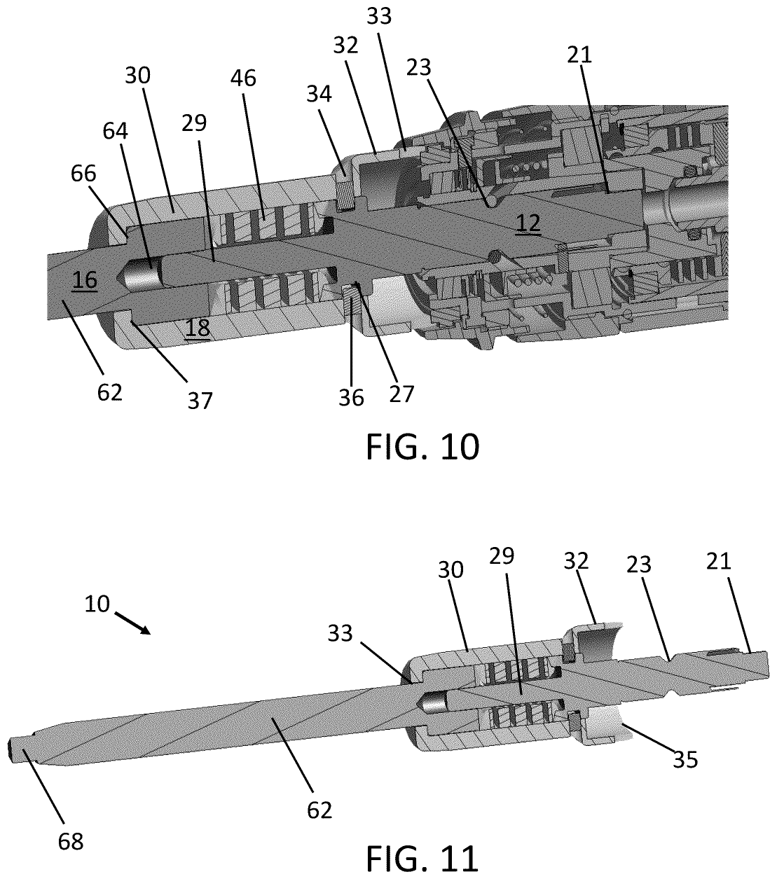

When the clutch assembly of torque limiter 10 and housing 18 are assembled, fourth gear 60 of torque shaft 16, one-piece clutch 14, and a portion of quick connect 12 are disposed within cavity 31 such that channel 27 is aligned pin holes 34 and shoulder 66 is adjacent to inner shoulder 37, as best shown in FIG. 10. With pin holes 34 aligned with channel 27, each of pins 36 are positioned within the channel and within one of the pin holes such that the pins extend from the channel an interior region of the pin holes. Each of pins 36 has a diameter that is slightly smaller than the width of channel 27 such that pins can slide through the channel but cannot move in a direction along the longitudinal axis of torque limiter 10. This results in a portion of the clutch assembly of torque limiter 10 being rotatably connected to housing 18 such that positions of the clutch assembly and the housing are fixed with respect to each other along the longitudinal axis of torque limiter 10. In this manner, the clutch assembly of torque limiter 10 may be rotatably connected to housing 18 such that the clutch assembly can freely rotate within cavity 31 and first openings 33 and second openings 35 while the housing remains stationary.

During operation of the torque limiter, housing 18 of torque limiter 10 holds torque shaft 16 and quick connect 12 with respect to the longitudinal axis of the torque limiter 10 while one-piece clutch 14 compresses and shifts therebetween in response to a maximum torque being applied. The compression of one-piece clutch 14 allows gear teeth to disengage. When compressed or flexed, one-piece clutch 14 is elastically deformed such that the one-piece clutch can expand back to its original form similar to a compression spring to reengage gear teeth. In this manner, one-piece clutch 14 can absorb torsional force from the power drill by deforming (i.e., flexing or compressing like a compression spring) and slipping between the gear teeth of torque shaft 16 and/or quick connect 14 to prevent over-tightening of a bone screw and/or locking cap.

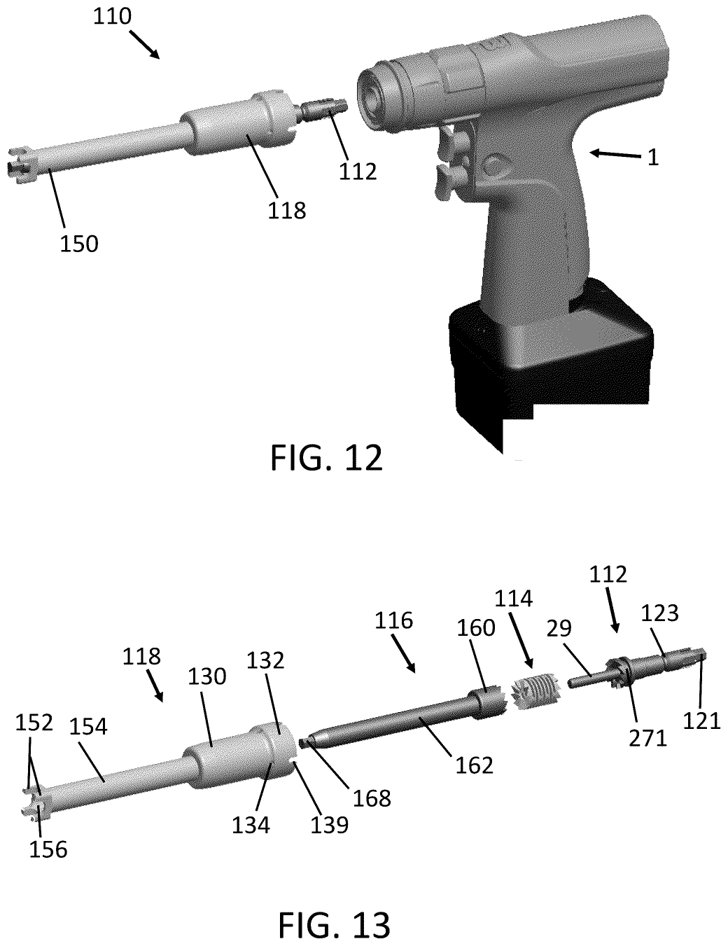

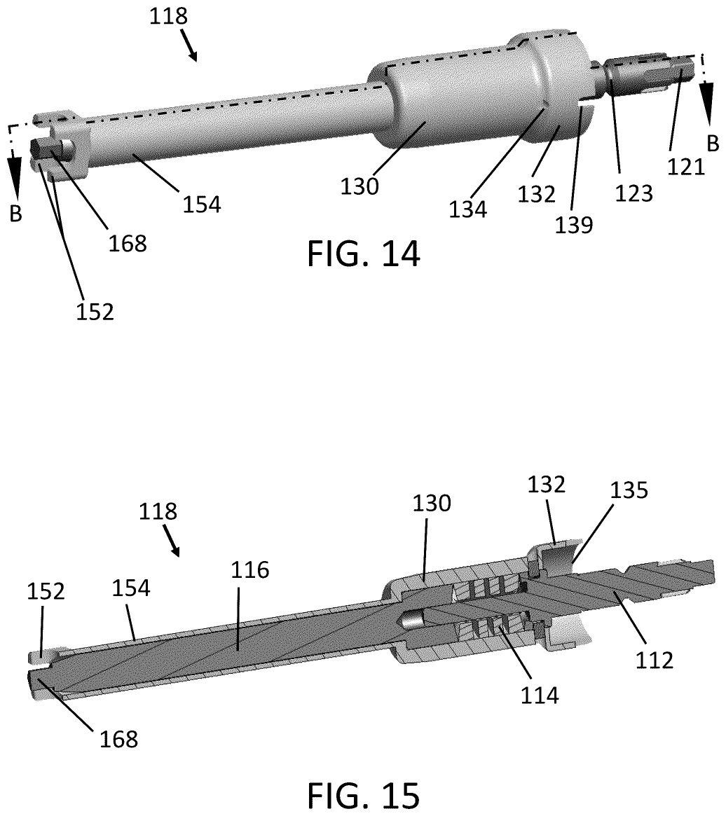

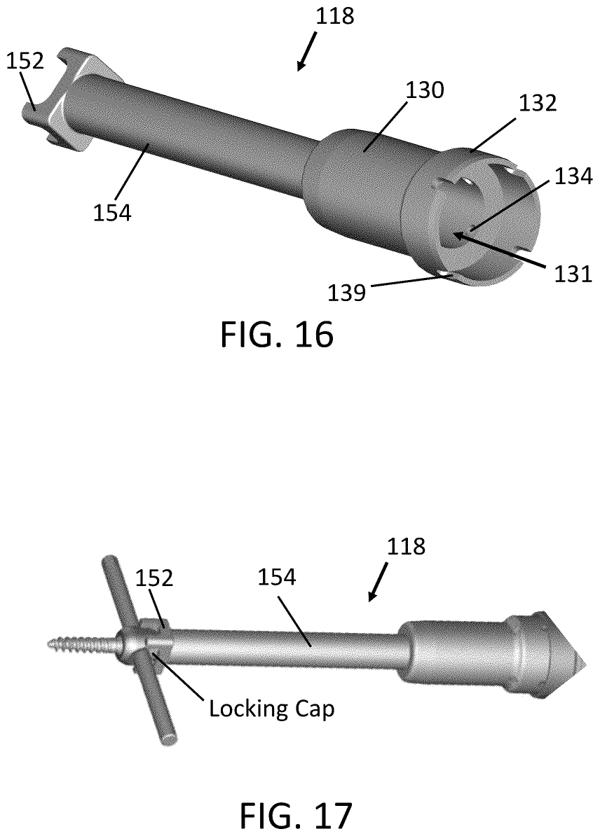

In other embodiments, housing 118 includes an anti-torque key 150 that extends distally from the housing and is axially aligned therewith, as shown in FIGS. 12-15. Anti-torque key 150 includes multi-pronged tip 152 and sleeve 154 that extends from cover portion 130 of housing 118. Sleeve 154 defines an aperture 156 located at the distal end of a hollow tube 158 that is in communication with cavity 131 of housing 18. Hollow tube 158 is constructed to receive torque shaft 116 such that the torque shaft can rotate within the hollow tube without restriction or contact with the inner surface of sleeve 154. Multi-pronged tip 152 has four prongs extending distally from a platform positioned perpendicular to the longitudinal axis of the torque limiter and around aperture 156, the prongs extend no further than tip 168 of torque shaft 116. Anti-torque key 150 and housing 118 form one solid body that encases a majority of the clutch assembly of torque limiter 110. However, in some embodiments, anti-torque key 150 may be threadably attached to housing 118. In some embodiments, housing 118 may include sleeve 154 without multi-pronged tip 152. Multi-pronged tip 152 may be detachable connected to sleeve 154 (e.g., threadably attached thereon). Multi-pronged tip 152 may be shaped to cooperate with a pedicle screw tulip, as shown in FIG. 17, or have a modular design thereby allowing anti-torque key 150 to be used with a variety of tulips, locking caps, and/or screws.

In another aspect, the present disclosure relates to a kit that may include a torque limiter instrument having a one-piece spring clutch, such as one-piece clutch 14, that is encased in a non-rotating housing cover, such as housing 18, capable of connecting to a power drill. The kit may include a torque shaft and a quick connect drive that are assembled or can be assembled with a one-piece clutch. The housing cover in the kit may include an anti-torque key extending therefrom. The kit may further include replacement parts such as additional one-piece clutches that have various configurations. For example, multiple interchangeable one-piece clutches that are each configured to withstand different amounts of maximum applied torque may be included in a kit and thereby expand the usability of the torque limiter instrument. Additionally, the kit may have multiple torque shafts that are interchangeable and have different lengths, and/or multiple torque shafts that have different tips designed to be used for different bone screws or locking caps.

In yet another aspect, the present disclosure relates to a method of assembling a torque limiter having a one-piece clutch. For example, torque limiter 10 may be assembled by combining first gear 25 of quick connect 12 with second gear 40 of one-piece clutch 14 such that the planar surfaces of the first gear contact the angled surfaces of the second gear, as shown in FIG. 4. Next, third gear 42 of one-piece clutch 14 is combined with fourth gear 60 of torque shaft 16 such that the angled surfaces of the fourth gear are in contact with the angled surfaces of the third gear. Combining the aforementioned gear teeth can be done in any order. Once quick connect 12, one-piece clutch 14, and torque shaft 16 have all been combined, inserting rod 62 of the torque shaft into first opening 33 of housing 18 and sliding the housing over the rod until shoulder 66 of the torque shaft contacts inner shoulder 37 of the housing such that pin holes 34 are aligned with channel 27 of quick connect 12. Finally, inserting one of pins 36 into each of pin holes 34 such that each of the pins has one end within channel 27 and another end within one of the pin holes. If housing 18 includes anti-torque key 50, then rod 62 would be inserted through first opening 33 and then into sleeve 54.

In another aspect, the present disclosure relates to a method of replacing the one-piece clutch with a replacement one-piece clutch which may require partially disassembling the torque limiter. For example, torque limiter 10 may be partially disassembled and one-piece clutch may be replaced with a replacement one-piece clutch by removing pins 36 from channel 27 of quick connect 14. Pins 36 may have threads such that they may be threaded into and out of pin holes 34 allowing for quick assembly and disassembly of torque limiter 10. Alternatively, pins 36 may be press fit pins.

Once pins are removed, removing quick connect 12 and one-piece clutch 14 from housing 18. After torque limiter 10 has been partially disassembled, the replacement one-piece clutch may be deployed by inserting the replacement one-piece clutch into housing 18 such that the teeth of the third gear (i.e., distally facing gear) of the replacement one-piece clutch engage and tightly fitted against the gear teeth of fourth gear 60 of torque shaft 16. Once the replacement one-piece clutch is inserted and properly positioned, inserting quick connect 12 into housing 18 such that alignment shaft 29 is inserted through a hollow annulus of the replacement one-piece clutch and into alignment hole 64 of torque shaft 16 until the gear teeth of first gear 25 engage and tightly fitted against the teeth of the second gear (i.e., proximally facing gear) of the replacement one-piece clutch. After quick connect 12 has been inserted and properly positioned such that pin holes 34 align with channel 27, reinstalling pins 36 by inserting the pins into their respective pin holes 34 until they are received by channel 27 of the quick connect. The replacement one-piece clutch may be also replaced with another one-piece clutch by repeating the same steps. The replacement one-piece clutch may be configured to withstand a different maximum torque than that of one-piece clutch 10 due to having a different structural configuration or being made from a different material. In this manner, torque limiter 10 may be customized as necessary to limit different values of applied torque by simply replacing one-piece clutch 14 with another interchangeable one-piece clutch. This may allow medical personal to adjust the torque limiter either before or during a surgical procedure.

In another aspect, the present disclosure relates to a method of attaching a torque limiter having a one-piece clutch to a power drill. For example, a torque limiter, such as torque limiter 10, may be attached to a power drill by inserting and engaging a quick connect component, such as quick connect 12, into a tool hole of the power drill. While inserting the quick connect component, securing a non-rotating housing, such as housing 18, against a surface of the power drill by inserting slots on housing around nubs on the power drill. In some embodiments, the housing may be inserted into a slot located on the exterior of the power drill to provide a sturdy connection between the power drill and non-rotating housing. In this manner, users are provided with better control over the torque limiter as they are given the ability to directly hold the torque limiter during deployment. The non-rotating housing is also advantageous because it protects users by preventing gloves and other surgical paraphernalia from getting caught in the rotation components of the torque limiter.

In another aspect, the present disclosure relates to a method of tightening bone screws and/or locking caps of pedicle screws with a tool that includes a torque limiter having a one-piece clutch, a quick connect, and a torque shaft. For example, a bone screw may be tightened by the torque limiter connected to a power drill by inserting and engaging a tip of the torque shaft, such as tip 68 of torque shaft 16, into the head of a bone screw having a recess corresponding to the shape of the tip. Once the tip has engaged the head of the bone screw, operating the power drill such that torsional force is transferred from the power drill to the bone screw via the torque limiter. When the torque applied to the bone screw exceeds a maximum threshold, deforming the one-piece clutch such that the gear teeth of the one-piece clutch shift along the gear teeth of either the quick connect, the torque shaft, or both which limits or prevents certain levels of torque from being applied to the bone screw. Once the one-piece clutch is compressed due to excessive torque, the gear teeth of the one-piece cutch reset and reengage with the quick connect and torque shaft such that torque actuation can continue.

In some embodiments, when the drill is activated to spin in a first direction (e.g., clockwise) to tighten a bone screw or locking cap, the quick connect may transfer torque to the one-piece clutch. Once the maximum torque is achieved, the distal gear teeth of the one-piece clutch may skip between the teeth of gear on the torque shaft. When the power drill is activated to spin in a second direction (e.g., counterclockwise), the quick connect continues to transfer torque to the one-piece clutch. Once maximum torque is achieved, the gear teeth of the one-piece clutch slips between the gear teeth of the quick connect. In this manner, the one-piece clutch can transfer torque and limit torque in both rotational directions (i.e., clockwise and counterclockwise).

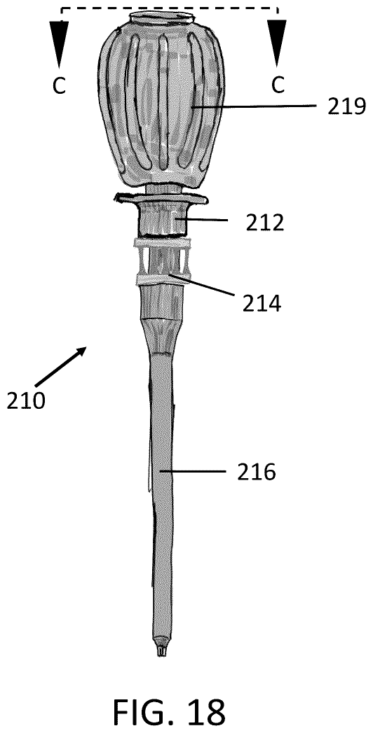

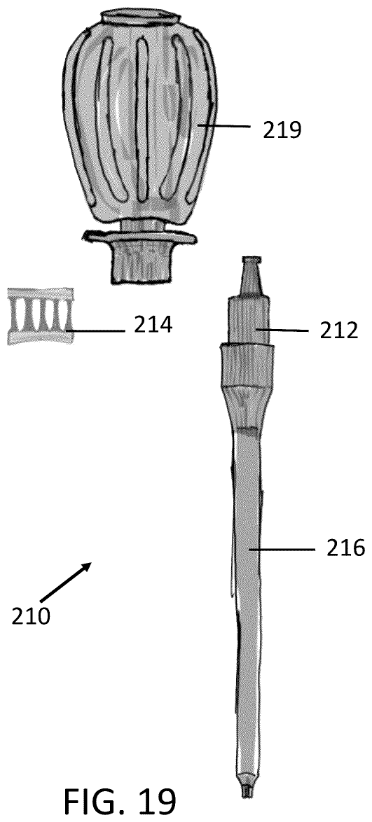

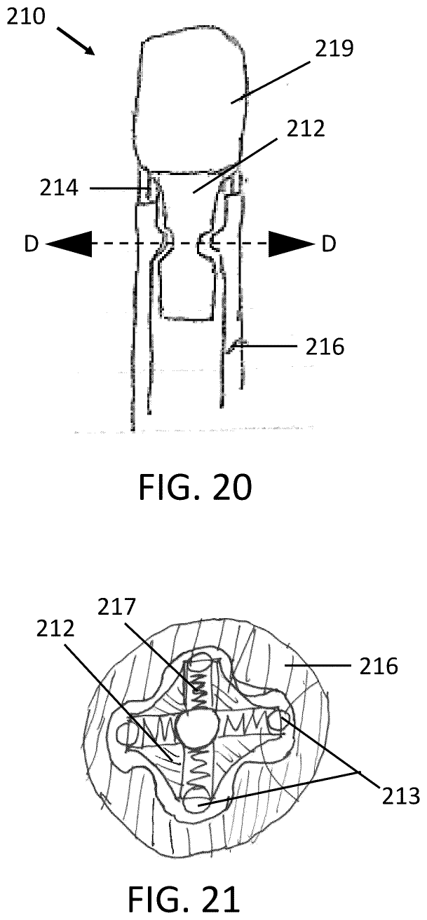

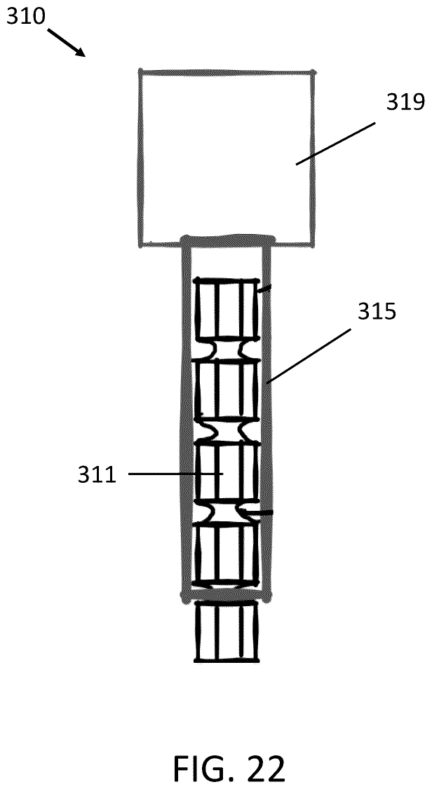

In another aspect, as shown in FIGS. 18-21, the present disclosure relates to a torque limiter that is configured to attach to a handle such that the torque limiter may be used for manual tightening. Torque limiter 210 may include a torque shaft 216, a break-away support 214, a quick connect 212 that is inserted through the breakaway support and into the torque shaft, and a handle 219. Torque limiter 210 operates such that breakaway support 214 prevents rotation between quick connect 212 and torque shaft 216. In other words, torque is transferred from quick connect 212 to torque shaft 216 through breakaway support 214. Once maximum torque is achieved, breakaway support 214 breaks and quick connect 212 rotates within torque shaft 216. In this manner, breakaway support 214 limits applied torque and thereby prevents overtightening of a screw or locking cap.

In some other embodiments, quick connect 212 may be configured to detachably connect to handle 219, e.g., threadably attach. Quick connect 212 may also include rollers 213 that are pushed radially outward by springs 217, as shown in FIG. 21. Rollers 213 fit into grooves disposed in the inner surface of torque shaft 216. Rollers 213 may be positioned at a narrow region along the hollow interior of torque shaft 216 to increase the engagement between the rollers and the grooves of the torque shaft. The corresponding fit between the rollers 213 resist rotation of quick connect 212 while within torque shaft 216. Once a certain torque value is achieved, springs 217 are compressed as rollers 213 are pressed into quick connect 212 which allows the quick connect to rotate within the grooves of torque shaft 216. In this manner, rollers 213 may be use along with the breakaway support 214 to transfer and limit torque during manual tightening of screws or locking caps.

In another aspect, as shown in FIG. 21, the present disclosure relates to another torque limiter used for manual tightening. Torque limiter 310 may include handle 319, hollow shaft 315 extending from the handle, and breakaway torque shaft 311. Breakaway torque shaft 311 includes a series of tip components interconnected by a narrow neck, each tip component is configured to engage a screwhead for tightening when it because the most distal tip component. Hollow shaft 315 is configured to receive breakaway torque shaft 311 such that the breakaway torque shaft cannot rotate therein and at least one tip component remains external to the hollow shaft at a distal end. Torque is transferred from hollow shaft 315 to a screw through breakaway torque shaft 311. Once maximum torque is achieved, the narrow neck between the two most distal tip components breaks, and the most distal tip component detaches from the remainder of breakaway torque shaft 311. The maximum applied torque needed to break the narrow neck of breakaway torque shaft 311 is determined by the material and geometrical dimensions of the narrow neck. Hollow shaft 315 then releases breakaway shaft 311 to allow the next tip component to drop out of the hollow shaft such that it can engage a screw head for tightening. In this manner, the narrow neck of breakaway torque shaft 311 limits applied torque to prevent overtightening and provides a replacement tip for further tightening after the most distal tip component breaks off.

It is to be understood that the disclosure set forth herein includes any possible combinations of the particular features set forth above, whether specifically disclosed herein or not. For example, where a particular feature is disclosed in the context of a particular aspect, arrangement, configuration, or arrangement, that feature may also be used, to the extent possible, in combination with and/or in the context of other particular aspects, arrangements, configurations, and arrangements of the technology, and in the technology generally.

Furthermore, although the technology herein has been described with reference to particular features, it is to be understood that these features are merely illustrative of the principles and applications of the present technology. It is therefore to be understood that numerous modifications, including changes in the sizes of the various features described herein, may be made to the illustrative arrangements and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. A torque limiter comprising:
a drive shaft having first teeth;
a one-piece clutch having a top surface defining second teeth and a bottom surface defining third teeth, the second and the third teeth being integral to a flexible region of the one-piece clutch, wherein the one-piece clutch defines a single structural unit; and
a torque shaft having fourth teeth,
wherein the first teeth engage the second teeth and the third teeth engage the fourth teeth.

2. The torque limiter of claim 1, further comprising a housing disposed around portions of the drive shaft, the torque shaft, and the one-piece clutch.

3. The torque limiter of claim 2, wherein the housing encases the first, the second, the third, and the fourth teeth.

4. The torque limiter of claim 3, wherein the housing includes an anti-torque key.

5. The torque limiter of claim 4, wherein the housing includes a plurality of slots disposed radially on a proximal edge of the housing.

6. The torque limiter of claim 5, wherein the housing includes a plurality of pin holes that are disposed around the housing and configured to align with a channel in the drive shaft, the channel being adjacent to the first teeth.

7. The torque limiter of claim 6, further comprising a plurality of pins that are disposed partially within the channel of the drive shaft and partially within the plurality of pin holes of the housing.

8. The torque limiter of claim 7, wherein at least one of the plurality of pins sits below an outer surface of the housing that surrounds each of the plurality of pin holes.

9. The torque limiter of claim 5, wherein the plurality of slots are configured to engage with segments of a power drill.

10. The torque limiter of claim 1, wherein the one-piece clutch is configured to transfer an applied torque from the drive shaft to the torque shaft up to a maximum torque.

11. The torque limiter of claim 10, wherein the one-piece clutch is configured to compress or flex under an amount of torque that exceeds the maximum torque.

12. The torque limiter of claim 11, wherein the compressing or flexing of the one-piece clutch is configured to allow the second teeth or the third teeth to slip with respect to the first teeth or the fourth teeth, respectively.

13. The torque limiter of claim 1, wherein the one-piece clutch is additively manufactured.

14. The torque limiter of claim 1, wherein the one-piece clutch further includes
a plurality of pillars integrally formed between a plurality of layers that extend perpendicular to a longitudinal axis of the torque limiter.

15. The torque limiter of claim 14, wherein the plurality of pillars extend parallel to the longitudinal axis of the torque limiter.

16. The torque limiter of claim 15, wherein the one-piece clutch is a cylinder and further includes a central aperture extending through both the second and third teeth.

17. The torque limiter of claim 16, wherein the drive shaft includes an alignment shaft extending from the first teeth along the longitudinal axis of the torque limiter, the alignment shaft configured to extend through the central aperture of the one-piece clutch and into a central hole in the torque shaft.

18. A kit comprising:
a drive shaft having first teeth;
a torque shaft having second teeth; and
a plurality of one-piece clutches each having a top clutch face having a third teeth and a bottom clutch face having a fourth teeth, each one-piece clutch is configured to transmit a different maximum torque, wherein the first teeth is adapted to engage the third teeth and the second teeth is adapted to engage the fourth teeth.

19. The kit of claim 18, further comprising a powered handpiece.

20. The kit of claim 18, further comprising a housing configured to enclose a portion of the drive shaft, the torque shaft, and at least one of the plurality of one-piece clutches in an assembled arrangement.

21. The kit of claim 20, further comprising pins configured to rotatably attach the housing and the drive shaft such that the drive shaft, the torque shaft, and one of the plurality of one-piece clutches remain assembled together.

22. A surgical instrument for tightening surgical hardware, comprising:
a housing;
a torque shaft;
a drive shaft disposed partially within the housing and rotatably connected to the housing; and
a deformable clutch defining a single part having first gear teeth and second gear teeth facing away from each other, the deformable clutch disposed between the drive shaft and the torque shaft.
